(19) United States
(12) Patent Application Publication
(10) Pub. No.: US 2026/0124168 A1
(43) Pub. Date: May 7, 2026

(54) OIL COMPOSITION COMPRISING CAPROIC ACID RESIDUES, UNSATURATED FATTY ACID RESIDUES AND SATURATED FATTY ACID RESIDUES

(71) Applicant: AAK AB (PUBL), Malmö (SE)

(72) Inventors: Lotte SMOLDERS, Malmö (SE); Kim OLOFSSON, Malmö (SE); Martin JOHANSSON, Malmö (SE); Johanna ROSENQVIST, Malmö (SE)

(21) Appl. No.: 19/117,146

(22) PCT Filed: Oct. 6, 2023
(86) PCT No.: PCT/SE2023/051002

§ 371 Date: Mar. 31, 2025

(30) Foreign Application Priority Data
Oct. 6, 2022 (SE) 2230323-4

(51) Int. Cl. A61K 31/23; A23D 9/00; A23D 9/04; C11C 3/00

(52) U.S. Cl. CPC A61K 31/23; A23D 9/00; A23D 9/04; C11C 3/00

(57) ABSTRACT

An oil composition comprises from 1% to 20% by weight of caproic acid residues (C6:0); at least 55% by weight of C16 to C18 unsaturated fatty acid resides; and less than 40% by weight of C8 to C24 saturated fatty acid residues; wherein said percentages of fatty acid residues refers to fatty acids bound as acyl groups in glycerides in the oil composition and being based on the total weight of saturated and unsaturated C4 to C24 fatty acid residues bound as acyl groups present in the oil composition; and wherein at least 50% of the caproic acid residues present in the oil composition are present in a triglyceride comprising at least one caproic acid residue and at least one fatty acid residue comprising from 16 to 18 carbon atoms.

### FIELD OF THE INVENTION

The invention relates to oil compositions and nutritional products comprising said compositions. The invention also relates to methods of supplementing the diet of individuals with said nutritional products and oil compositions.

### BACKGROUND OF THE INVENTION

It is known that short chain fatty acids (SCFA) are a desirable component of the diet of mammals. Short chain fatty acids are fatty acid molecules containing typically six or less carbon atoms. Examples of SCFA include acetic acid, propionic acid, butyric acid, isobutyric acid, valeric acid and caproic acid. SCFA are reported as being associated with various health and wellbeing benefits. For example, SCFA have been reported as being associated with healthy gastrointestinal function and intestinal absorption, a healthy immune system, reduced chronic inflammation and improved metabolism. It has also been suggested that circulating SCFA may be associated with improved weight and appetite regulation thereby potentially being used as an obesity preventative. Circulating SCFA levels are also reported as being associated with regulation of blood sugar levels and the prevention of insulin resistance meaning that SCFA could potentially be used to prevent or ameliorate the development of type II diabetes. Blaak et al., Short chain fatty acids in human gut and metabolic health, Beneficial Microbes, 2020; 11(5): 411-455 discloses that actetate, butyrate and propionate may play a role in the pathophysiology of gastrointestinal health, obesity and type II diabetes. Canfora et al., Short-chain fatty acids in control of body weight and insulin sensitivity, Endocrinology, Volume 11, October 2015 also discusses the link between actetate, butyrate and propionate and obesity and type II diabetes.

Whilst SCFA in general are known to be associated with the positive effects discussed above, the prior art is mainly focused upon the study of the in vivo effects of butyric acid (C4:0), propionic acid (C3:0) and acetic acid (C2:0), with the principal focus being on butyric acid (C4:0). For example, Steliou et al., Butyrate Histone Deacetylase Inhibitors, BioResearch Open Access, Volume 1, Number 4, August 2012 discusses how butyrate is a histone deacetylase inhibitor that can potentially be used to treat diet induced obesity and prevent insulin resistance. Ivanov et al., The role of prenatal melatonin in the regulation of childhood obesity, Biology, 2020, 9, 72 discloses that butyrate may regulate obesity by being active on the p-opioid receptor. Lin et al., Butyrate and propionate protect against diet-induced obesity and regulate gut hormones via free fatty acid receptor 3-independent mechanisms, Plos One, April 2012, Volume 7, Issue 4, e35240 discusses how butyrate and propionate protect against diet induced obesity and insulin resistance and that these SCFA may have a major impact upon therapies for diabetes type II and obesity. Whilst the art has investigated the potential and mechanism of action of butyric acid (and to a lesser extent propionic and acetic acid) as a supplement for promotion of the abovementioned health benefits, caproic acid (C6:0) has to date not been focused upon.

It is known in the art that where butyric acid is to be provided as an orally administered nutritional supplement, providing the fatty acid in a free fatty acid form is not desirable since the butyric acid is broken down in the stomach and does not reach the systemic circulation where it can promote the health benefits discussed above. Providing the butyric acid in the form of a salt such as sodium butyrate is also undesirable since it has an unpleasant flavour and odour meaning that it is less organoleptically acceptable for consumers when orally ingested.

In order to alleviate these problems, several attempts have been made to provide the butyric acid in the form of a plant derived triglyceride oil. However, in order to provide a butyrate-containing triglyceride oil with sufficient organoleptic properties for oral consumption, it has been found necessary to subject the plant derived triglycerides to a deodorization process step in order to remove odiferous compounds that impart an unpleasant smell, colour or taste to the triglycerides. Deodorization is a normal process step in the refining of plant derived triglyceride oils once they have been extracted from plant sources. Deodorization steps typically involve subjecting the triglyceride oil to high temperatures of from 100° C. to 260° C. whilst under vacuum.

It has been appreciated by the inventors of the present invention that when deodorization of butyrate-containing triglyceride oils (and also acetate or propionate-containing triglyceride oils) is carried out, significant quantities of the triglyceride oil are lost during the deodorization step. During deodorization, the oils are typically heated to high temperatures. This heating process can cause the triglycerides to evaporate which reduces the yield of the deodorization process of the triglyceride oil. This is undesirable when manufacturing the oils on a large scale as the reduced yield negatively impacts manufacturing process economics.

The inventors have thus appreciated that there remains a continued need in the art for nutritional products for oral consumption comprising triglyceride oil compositions that can provide SCFA to the systemic circulation and that address the problems discussed above.

### SUMMARY OF THE INVENTION

The present invention is based on the surprising finding that certain triglyceride oil compositions address or alleviate the problems discussed above. It has surprisingly been found that certain triglyceride oil compositions that contain caproic acid have a higher yield after being subjected to a deodorization step than analogous triglyceride compositions comprising butyric acid instead of caproic acid. Without being limited by theory, this is believed to be due to the higher average molecular weight of triglycerides that contain caproic acid instead of the shorter chain butyric acid. It is believed that the higher average molecular weight of the triglycerides means that the triglycerides are less susceptible to evaporation and loss during the deodorization process step which is important to carry out so that the triglyceride oil has acceptable organoleptic properties. The certain oil compositions can thus be produced and deodorized at higher yield providing improved economics to manufacturing processes. Caproic acid (C6:0) when delivered to the systemic circulation can also provide the same or similar health and wellbeing benefits as shorter chain SCFCs such as butyric acid.

According to an aspect of the invention, there is provided an oil composition comprising from 1% to 20% by weight of caproic acid residues (C6:0); at least 55% by weight of C16 to C18 unsaturated fatty acid residues; and less than 40% by weight of C8 to C24 saturated fatty acid residues; wherein said percentages of fatty acid residues refers to fatty acids bound as acyl groups in glycerides in the oil composition and being based on the total weight of saturated and unsaturated C4 to C24 fatty acid residues bound as acyl groups present in the oil composition; and wherein at least 50% of the caproic acid residues present in the oil composition are present in a triglyceride comprising at least one caproic acid residue and at least one fatty acid comprising from 16 to 18 carbon atoms.

The oil composition is a triglyceride oil composition. Typically, at least 50% of the fatty acid moieties present in the oil composition are present in a triglyceride molecule.

Preferably at least 70%, more preferably at least 80%, still more preferably at least 90% and most preferably at least 95% of the fatty acid moieties present in the oil composition are present in a triglyceride molecule.

The terms “fat” and “oil” as used herein are used interchangeably to refer to glyceride fats and oils containing fatty acid acyl groups and does not imply any particular melting point.

The term “fatty acid”, as used herein, refers to straight chain saturated or unsaturated (including mono- and poly unsaturated) carboxylic acids having 4 to 24 carbon atoms. A fatty acid having x carbon atoms and y double bonds may be denoted Cx:y. For example, palmitic acid may be denoted C16:0, oleic acid may be denoted C18:1. Percentages of fatty acids in compositions referred to herein include acyl groups in tri-, di- and mono-glycerides present in the glycerides and are based on the total weight of C4 to C24 fatty acids. The fatty acid profile (i.e. composition) may be determined, for example, by fatty acid methyl ester analysis (FAME) using gas chromatography according to ISO 16958.

The terms hexanoic acid and caproic acid as used herein are used interchangeably to refer to the compound with the structural formula CH₃CH₂CH₂CH₂CH₂ (CO)OH. The oil composition preferably comprises caproic acid (C6:0) in an amount of from 1% to 15% by weight, more preferably from 1% to 10% by weight; and still more preferably from 2% to 15% by weight. Most preferably, the oil composition comprises caproic acid (C6:0) in an amount of from 2% to 10% by weight.

In some instances, the oil composition comprises from 4% to 20% by weight; such as from 4% to 15% by weight of caproic acid. For example, the oil composition may comprise from 4% to 10% by weight of caproic acid. In other instances, the oil composition comprises from 5% to 20% by weight; such as from 5% to 15% by weight of caproic acid. For example, the oil composition may comprise from 5% to 10% by weight of caproic acid.

Typically, the oil composition comprises from 2% to 30% by weight of butyric acid residues (C4:0). Preferably, the oil composition comprises butyric acid (C4:0) in an amount of from 2% to 25% by weight, more preferably from 2% to 20% by weight; and most preferably from 2% to 15% by weight.

In other preferable instances, the oil composition comprises butyric acid (C4:0) residues in an amount of from 7.5% to 30% by weight; preferably from 7.5% to 25% by weight; more preferably from 7.5% to 20% by weight and most preferably from 7.5% to 15% by weight.

In certain preferable instances, the oil composition comprises from 4% to 20% by weight of caproic acid residues and from 7.5% to 30% by weight of butyric acid residues; such as from 4% to 10% by weight caproic acid residues and from 7.5% to 15% by weight of butyric acid residues.

Typically, at least 50% of the butyric acid residues present in the oil composition are present in a triglyceride comprising at least one butyric acid residue and at least one fatty acid residue comprising from 16 to 18 carbon atoms.

An advantage of including butyric acid in the compositions along with caproic acid is that the compositions may mimic and comprise the same fatty acids found in animal milk fat which is high in both caproic acid and butyric acid. The oil compositions of the invention may thus be used as milk fat substitute compositions. Since the oil compositions of the invention are typically non-animal derived, the compositions may advantageously serve as non-animal based milk fat alternatives. Advantageously, the oil compositions of the invention comprise higher amounts of unsaturated fatty acids and lower amounts of saturated fatty acids than milk fat. Typically, the oil compositions of the invention also comprise lower amounts of palmitic acid than milk fat. This is advantageous in view of the negative health effects associated with consuming too much saturated fat or too much palmitic acid which is believed to undesirably effect circulating cholesterol levels.

Another advantage of including butyric acid residues in the oil composition of the invention along with caproic acid residues is that there has surprisingly been found to be a synergistic increase in expression of the anti-inflammatory biomarker IL-10, as shown in the examples below. The combination of caproic acid (C6:0) and butyric acid (C4:0) has been found to synergistically increase expression levels of IL-10 which is associated with amelioration and prevention of chronic inflammation and obesity.

Typically, the oil composition has a weight ratio of butyric acid residues (C4:0) to caproic acid residues (C6:0) of from 1:1 to 6:1, preferably from 1:1 to 4:1, more preferably from 1:1 to 2.5:1 and most preferably from 1:1 to 2:1.

In the oil composition, at least 50% of the caproic acid residues present in the oil composition are present in a triglyceride comprising at least one caproic acid residue and at least one fatty acid residue comprising from 16 to 18 carbon atoms. An additional advantage of the invention is that it has surprisingly been found that where the caproic acid residues are included in a triglyceride molecule along with longer chain fatty acids with 16 to 18 carbon atoms, the cleavage of the caproic acid moiety from the triglyceride by lipases in the gastrointestinal tract is advantageously delayed until the triglyceride molecule is present in the small intestine. This is surprising since it has also been found by the inventors that triglycerides containing three SCFCs such as three caproic acid residues or a mixture of caproic acid and butyric acid residues are broken down in the stomach by gastric lipases after oral ingestion. It is undesirable that the caproic acid is cleaved from the triglyceride molecule in the stomach since it is believed that where this occurs the caproic acid does not find its way into the systemic circulation and is instead absorbed and metabolised by stomach cells which use the caproic acid as an energy source. In this scenario the caproic does not reach the systemic circulation and so cannot promote the advantageous health effects discussed above. In contrast, where the cleavage of the caproic acid from the triglyceride is delayed until the small intestine as is the case with compositions of the invention, the free caproic acid residues are available to be absorbed in the small intestine where they then enter the systemic circulation. It is believed that the presence of a fatty acid residue comprising 16 to 18 carbon atoms in the triglyceride molecule along with the caproic acid residue inhibits the action of gastric lipases that would cleave the caproic acid from the triglyceride.

It is particularly preferred that in the oil compositions of the invention that the caproic acid residues and butyric acid residues if present are present in triglycerides comprising two fatty acids comprising from 16 to 18 carbon atoms. It is believed that the caproic acid/butyric acid cleavage from the glycerol backbone of the triglyceride by gastric lipases is inhibited even more when the triglyceride comprises two fatty acids comprising from 16 to 18 carbon atoms, compared to when the triglycerides comprise only one fatty acid residue comprising 16 to 18 carbon atoms. The inclusion of such triglycerides in the composition can thus provide an even higher intestinal absorption (and thus circulating plasma concentration) of the short chain fatty acids. This effect is observed in the examples discussed below, where compositions comprising higher amounts of triglycerides comprising one caproic acid residue and two fatty acids with 16 to 18 carbon atoms provide higher post prandial caproic acid plasma concentration than compositions comprising lower amounts of said triglycerides, despite said compositions comprising lower amounts of said triglycerides comprising overall higher amounts of caproic acid residues.

Typically, at least 60%, preferably at least 70%, and more preferably at least 75% of the caproic acid residues present in the oil composition; and at least 60%, preferably at least 70%, and more preferably at least 75% of butyric acid residues present in the oil composition are present in a triglyceride comprising at least one butyric acid or caproic acid residue and at least one fatty acid comprising 16 to 18 carbon atoms.

Preferably, at least 80% of the caproic acid residues present in the oil composition; and at least 80% of the butyric acid residues present in the oil composition are present in a triglyceride comprising at least one butyric acid or caproic acid residue and at least one fatty acid comprising 16 to 18 carbon atoms.

Typically, at least 20%, preferably at least 25%, and more preferably at least 30% of the caproic acid residues present in the oil composition; and at least 20%, preferably at least 25%, and more preferably at least 30% of butyric acid residues present in the oil composition are present in a triglyceride comprising a butyric acid or caproic acid residue and two fatty acid residues comprising 16 to 18 carbon atoms.

In a highly preferred instance, at least 25% of the caproic acid residues present in the oil composition and at least 25% of butyric acid residues present in the oil composition are present in a triglyceride comprising a butyric acid or caproic acid residue and two fatty acid residues comprising 16 to 18 carbon atoms. Still more preferably, at least 30% of the caproic acid residues present in the oil composition and at least 30% of butyric acid residues present in the oil composition are present in a triglyceride comprising a butyric acid or caproic acid residue and two fatty acid residues comprising 16 to 18 carbon atoms.

In these specific instances, preferably, at least 70% of the caproic acid residues present in the oil composition and at least 70% of butyric acid residues present in the oil composition are present in a triglyceride comprising at least one butyric acid or caproic acid residue and at least one fatty acid residue comprising 16 to 18 carbon atoms.

In some instances, at least 50%, such as at least 60%, at least 70%, or at least 80% of the caproic acid residues present in the oil composition; and at least 50%, such as at least 60%, at least 70%, or at least 80% of the butyric acid residues present in the oil composition are present in a triglyceride comprising a butyric acid or caproic acid residue and two fatty acid residues comprising 16 to 18 carbon atoms.

In the oil compositions of the invention, the percentage of caproic acid and/or butyric acid residues present in the oil composition that are present in a triglyceride comprising one or two fatty acid residues with 16 to 18 carbon atoms and butyric acid or caproic acid residues is determined according to ISO 17383, modified to include triglycerides with short chain fatty acids, using the following triglyceride standards for response factors: C12 (tributyrin), C18 (tricaproin), C24 (tricaprylin), C30 (tricaprin), C36 (trilaurin), C42 (trimyristin), C48 (tripalmitin) and C54 (tristearin).

Typically, the oil composition comprises fatty acid residues having from 16 to 18 carbon atoms in an amount of from 60% to 95% by weight, preferably from 65% to 90% by weight, more preferably from 70% to 85% by weight. As discussed above, it is important that the oil composition comprises a sufficient quantity of these fatty acids so that most of the caproic acid and butyric acid residues if present are present in triglycerides containing at least one fatty acid moiety having from 16 to 18 carbon atoms such that the cleavage of the caproic acid/butyric acid moieties by gastric lipases is inhibited. Typically, the oil composition has a weight ratio of fatty acid residues having 16 to 18 carbon atoms to butyric acid residues (C4:0) and caproic acid residues (C6:0) of from 1:1 to 20:1, preferably from 1.5:1 to 10:1, more preferably from 2:1 to 7:1 and most preferably from 2:1 to 6:1.

Typically, the majority of the fatty acid moieties having 16 to 18 carbon atoms present in the oil composition are unsaturated fatty acid moieties. This is advantageous as unsaturated fatty acids are typically considered healthier than saturated fatty acids. A diet too high in saturated fatty acids is associated with development of heart disease and unhealthy cholesterol levels. An advantage the oil compositions of the invention have over milk fat is the much lower levels of saturated fatty acids present and the much higher levels of unsaturated fatty acids. A further advantage of containing more unsaturated fatty acids is that the oil compositions of the invention tend to have lower melting points than compositions with higher saturated fatty acid contents. This may mean that the oil compositions of the invention are easier to include in liquid nutritional supplements (e.g. nutritional supplement drinks) and be more palatable and organoleptically acceptable for the consumer. Accordingly, typically, the oil composition comprises C16 to C18 unsaturated fatty acid residues in an amount of at least 60% by weight, preferably from 65 to 92% by weight, more preferably from 70 to 85% by weight, and most preferably from 75 to 85% by weight.

Typically, the oil composition comprises from 10% to 40% by weight of total saturated fatty acid residues; preferably from 15% to 40% by weight of total saturated fatty acid residues; and more preferably from 15% to 30% by weight of total saturated fatty acid residues.

As discussed above, the oil composition comprises less than 40% by weight of C8 to C24 saturated fatty acid residues. Preferably, the oil composition comprises less than 35% by weight of C8 to C24 saturated fatty acid residues; more preferably less than 30% by weight and most preferably less than 20% by weight.

Typically, the oil composition comprises 10% by weight or less; preferably 8% by weight or less; more preferably 6% by weight; and most preferably 4% by weight or less of palmitic acid (C16:0). As well as being a saturated fatty acid, palmitic acid (C16:0) is also considered worse for health than other saturated fatty acids such as stearic acid (C18:0).

Accordingly, it is desirable for the oil composition to not contain too high an amount of palmitic acid. Palmitic acid is also a major component of palm oil and products derived from palm oil are often high in palmitic acid. It is preferred that the oil compositions of the invention are not derived from palm oil due to public perception of the environmental impact of palm oil production.

Of the fatty acid residues comprising from 16 to 18 carbon atoms present in the oil composition, it is preferred that most of these fatty acids are fatty acids with 18 carbon atoms. It is believed that the fatty acids with 18 carbon atoms are better at inhibiting cleavage of caproic acid and butyric acid from triglycerides by gastric lipase than fatty acids having 16 or 17 carbon atoms. Additionally, fatty acids having 17 carbon atoms are less commonly occurring in plant derived triglyceride oils and so if the oil composition is derived from plant derived oils such as vegetable oils, the oil compositions will typically contain very low amounts of fatty acids having 17 carbon atoms. Accordingly, typically, the oil composition comprises 10% by weight or less; preferably 7% by weight or less; and more preferably 5% by weight or less of a total amount of fatty acid residues comprising 16 or 17 carbon atoms.

It is thus preferred that the oil composition is very high in fatty acids having 18 carbon atoms, and more preferably very high in unsaturated C18 fatty acids. Typically, the oil composition comprises from 50% to 90% by weight of oleic acid (C18:1) residues, preferably 56% to 85% by weight, and more preferably from 56% to 76% by weight of oleic acid residues (C18:1). Typically, the oil composition comprises from 1% to 25% by weight of linoleic acid (C18:2) residues; preferably from 1% to 10% by weight; more preferably from 2% to 9% by weight and most preferably from 4% to 9% by weight of linoleic acid residues (C18:2). However, typically, the oil composition comprises 10% by weight or less; preferably 8% by weight or less; more preferably 6% by weight or less; and most preferably 4% by weight or less of stearic acid residues (C18:0) since it is preferred that the oil composition is low in saturated fatty acid residues.

It is preferred that the oil composition comprises higher amounts of oleic acid (C18:1) than fatty acids with a greater degree of unsaturation such as linoleic acid (C18:2) because oleic acid has greater oxidative stability compared to fatty acids with a greater degree of unsaturation.

It is preferred that the oil composition is a non-hydrogenated oil composition, and more preferably a non-hydrogenated non-animal derived oil composition such as a vegetable derived composition or a microbially derived composition. It is preferable that the oil composition has not been subjected to hydrogenation and that none of the fatty acid moieties present in the oil composition have been subjected to hydrogenation since the process of hydrogenation increases the amount of trans unsaturated fatty acids present. Trans unsaturated fatty acids are associated with negative health effects in consumers. Accordingly, preferably, the oil composition comprises less than 5% by weight of trans unsaturated fatty acids; more preferably less than 3% by weight of trans unsaturated fatty acids; and most preferably less than 1% by weight of trans unsaturated fatty acids.

It is preferred that the oil composition is non-animal derived or at least partially non-animal derived.

Typically, at least 50% of the fatty acid residues present in the oil composition are non-animal derived such as vegetable derived and/or microbially derived; preferably wherein at least 70%; and more preferably at least 90% of the fatty acid residues present in the oil composition are non-animal derived such as vegetable derived and/or microbially derived. Most preferably, all of the fatty acid residues present in the oil composition are non-animal derived such as vegetable derived and/or microbially derived.

In cases where the fatty acid residues present in the oil composition are both microbially derived and vegetable derived, certain fatty acids may originally be from vegetable sources and certain fatty acids may originally be from microbial sources. Esterification of these fatty acids with glycerol can then produce an oil composition where all fatty acids present are vegetable derived or microbially derived.

Accordingly, the terms vegetable derived or microbially derived as used herein in the context of the fatty acid residues present in the oil compositions are used to refer to the original source of the fatty acids present as part of triglycerides in the oil composition. For example, for an oil composition produced by chemical esterification, if all fatty acids present in said oil composition originated from a vegetable oil, then all fatty acids present in the oil composition are encompassed by the term vegetable derived, even though the final oil composition is prepared by a chemical reaction.

Typically, at least 50% of the glycerol backbones of triglycerides present in the oil composition are non-animal derived such as vegetable derived and/or microbially derived; preferably wherein at least 70%; and more preferably at least 90% of the glycerol backbones of triglycerides present in the oil composition are non-animal derived such as vegetable derived and/or microbially derived. Most preferably, all of the glycerol backbones of triglycerides present in the oil composition are non-animal derived such as vegetable derived and/or microbially derived.

The oil compositions of the invention can be made by any suitable methods known in the art. For example, the oil compositions or components thereof can be produced from interesterification, transesterification or esterification using appropriate process conditions for these reactions that are known in the art.

In the case of esterification, oil compositions can be produced directly by reacting glycerol with appropriate free fatty acids in a desired ratio to produce the desired oil compositions.

For example, glycerol can be reacted with caproic acid, butyric acid, unsaturated fatty acids such as oleic acid and any other fatty acids that it is desired to include in the oil compositions. In a similar reaction, glycerol can be reacted with mixtures of esters. For example, glycerol can be reacted with an ester containing oleic acid and an ester containing caproic acid. Suitable reaction conditions necessary to obtain the desired products will be apparent to the person skilled in the art given the benefit of the present disclosure. For example, temperatures in the range of from 30° C. to 80° C. are typically used if enzymatic processes are used and higher temperatures are used if no enzymes are used. Appropriate catalysts include enzymatic catalysts such as lipases or chemical catalysts.

In the case of transesterification, a triglyceride such as a triglyceride containing oleic acid can be transesterified with a mixture of free fatty acids that comprise caproic acid and butyric acid if desired. Alternatively, a triglyceride containing caproic acid and optionally butyric acid can be transesterified with free fatty acids that comprise e.g. oleic acid.

Transesterification reactions also include the reaction of triglycerides with non-glyceride esters such as the reaction of an ester containing oleic acid and a triglyceride containing caproic acid; or the reaction of an ester containing caproic acid and a triglyceride containing oleic acid. After the transesterification reactions have been carried out, any free fatty acid by-products can be separated from the triglyceride components by distillation. Suitable reaction conditions necessary to obtain the desired products will be apparent to the person skilled in the art given the benefit of the present disclosure. Typically, temperatures of from 30° C. to 80° C. are used and an enzymatic catalyst is used such as a position selective or non-position selective lipase.

In the case of interesterification, two triglyceride oils are mixed together and reacted so as to reconfigure which fatty acid residues are bonded to which glycerol molecules resulting in different triglyceride molecules. For example a triglyceride high in caproic acid and optionally butyric acid can be interesterified with a triglyceride high in oleic acid. Suitable reaction conditions necessary to obtain the desired products will be apparent to the person skilled in the art given the benefit of the present disclosure and are discussed in for example, Dijkstra, A. J. Interesterification. In: The Lipids Handbook 3^{rd} Edition, pages 285-300 (F. D. Gunstone, J. L. Harwood, and A. J. Dijkstra (eds.), Taylor & Francis Group LLC, Boca Raton, FL) (2007). Typically, either a chemical catalyst such as sodium methylate may be used or an enzymatic catalyst such as position selective or non-position selective lipases. Typically, temperatures of from 30° C. to 100° C. are used.

In certain instances, the oil compositions comprise an interesterified blend, wherein the blend comprises (a) a vegetable oil selected from sunflower oil, rapeseed oil, canola oil, safflower oil, soybean oil, algae oil, olive oil, high oleic acid versions thereof, or mixtures thereof, and (b) a triglyceride oil comprising at least 10% by weight of caproic acid residues. The vegetable oil of (a) is high in oleic acid and so the interesterification product comprises both caproic acid and oleic acid residues.

Preferably, the triglyceride oil (b) comprises at least 20% by weight of caproic acid residues. In some instances, the triglyceride oil (b) can comprise very high amounts of caproic acid such as at least 80% by weight or at least 90% by weight of caproic acid residues. For example, the triglyceride oil (b) can comprise tricaproin.

In instances where it is desired to include both caproic acid and butyric acid in the oil compositions, the oil (b) typically comprises at least 90% by weight of caproic acid and butyric acid residues. For example, the oil (b) can comprise from 20% to 40% by weight of caproic acid residues. Preferably, the triglyceride oil (b) comprises from 20% to 40% by weight of caproic acid residues and from 60% to 80% by weight of butyric acid residues.

The triglyceride oil (b) can be obtained by e.g. esterification. In some instances, the triglyceride oil (b) is microbially derived such as obtained by a microbial fermentation process. For example, the triglyceride oil (b) can comprise triglycerides that comprise both caproic acid and butyric acid. Alternatively or additionally, the triglyceride oil (b) can comprise a blend of caproic acid rich triglycerides and butyric acid rich triglycerides. For example, the triglyceride oil (b) can comprise a mixture of tricaproin and tributyrin.

In the instances described above, typically, the interesterified blend is an interesterified blend of from 60% to 95% by weight of vegetable oil (a) and from 5% to 40% by weight of triglyceride oil (b).

In certain instances where the abovementioned process is used to provide the oil composition of the invention, the oil composition further comprises a vegetable oil component (c) that has not been produced by interesterification, transesterification or esterification; preferably wherein the vegetable oil component (c) comprises sunflower oil, rapeseed oil, canola oil, safflower oil, soybean oil, algae oil, olive oil, high oleic acid versions thereof, or mixtures thereof. In these instances, preferably, the vegetable oil component (c) is present in the oil composition in an amount of from 1% to 35% by weight; and wherein the interesterified blend is present in the oil composition in an amount of from 65% to 99% by weight.

After the oil composition of the invention has been manufactured, the oil composition is typically refined in a manner typical of plant derived oils using refining processes known in the art. For example, the oil composition can be distilled, bleached and/or deodorized using techniques commonly known in the art. Any one or more of distillation, bleaching and/or deodorizing can be carried out. Preferably all of these steps are carried and more preferably distillation is carried out first followed by bleaching and deodorization. Distillation is typically used to remove components with lower boiling points from the composition such as free fatty acids. Bleaching is carried out to remove pigmentation from the composition and increase the organoleptic acceptability of the composition. Compositions derived from plants typically comprise many pigments that are used to give the plants from which the compositions are derived their colour. Deodorization is carried out so as to remove compounds that give the composition an unpleasant flavour and/or smell. Further details of preferred deodorization processes are provided below.

Preferably, the oil composition is a deodorized oil composition or comprises a deodorized component. For example, in some instances the entire oil composition may be subjected to deodorization. In other instances, a component of the oil composition (such as the interesterified blend discussed above) may be deodorized and mixed with an oil that has not been deodorized. In still further instances, all components of the oil composition may be deodorized separately before being mixed together to form the oil composition.

As discussed above, deodorization of plant derived oils is important to provide a composition that is organoleptically acceptable to the consumers of nutritional products comprising the oil composition. As also discussed above, it has been found by the inventors that deodorization of an oil composition comprising caproic acid residues provides a deodorized composition with higher yield than where an analogous composition not comprising caproic acid residues but instead comprising shorter chain fatty acids is deodorized. The term analogous as used in this context is used to refer to an identical oil composition with the exception that instead of comprising caproic acid residues comprises an equivalent weight percentage amount of shorter chain fatty acids such as acetic, propionic or butyric acid. In order to provide this improved deodorization process step yield, it is preferred that the caproic acid residues are present in the oil composition in higher molecular weight triglycerides (such as triglycerides with one or two longer chain fatty acid residues and one caproic acid residue).

According to a second aspect of the invention, there is provided a nutraceutical product comprising an oil composition according to the first aspect of the invention.

The term nutraceutical product as used herein is used to refer to any product intended for oral consumption that is intended to be a medically or nutritionally functional food or supplement. Preferably, the nutraceutical product is a food product, an infant formula, or a supplement such as a pill or capsule.

The nutraceutical product can comprise the oil composition in any suitable amount. Typically, the nutraceutical product comprises the oil composition in an amount of from 0.5% to 100% by weight.

In some instances, the nutraceutical product is in liquid form. For example, the nutraceutical product can be a drink, yoghurt or cream. Additional components of these products other than the oil composition will be apparent to the skilled person given the benefit of the present disclosure, as will the suitable amounts that these additional components are included in the products. Typically, where the nutraceutical product is a liquid product, the product comprises the oil composition in an amount of from 0.5% to 10% by weight.

In other instances, the nutraceutical product is in solid form such as a powder tablet, dough, bar or capsule. Additional components of these products other than the oil composition will be apparent to the skilled person given the benefit of the present disclosure, as will the suitable amounts that these additional components are included in the products. Typically, where the nutraceutical product is a solid product, the product comprises the oil composition in an amount of from 5% to 100% by weight.

The nutraceutical product can comprise the oil composition in any suitable amount. Typically, the product comprises the oil composition in an amount such that the product comprises from 100 mg to 5000 mg; preferably from 500 mg to 2500 mg; more preferably from 750 mg to 1750 mg; and most preferably from 1000 mg to 1750 mg of a total of butyric acid and caproic acid residues.

Typically, the product comprises the oil composition in an amount such that the product comprises from 100 mg to 5000 mg per serving; preferably from 500 mg to 2500 mg per serving; more preferably from 750 mg to 1750 mg per serving; and most preferably from 1000 mg to 1750 mg per serving of a total of butyric acid and caproic acid residues.

Typically, the product comprises the oil composition in an amount such that the product comprises from 100 mg to 1000 mg; preferably from 200 mg to 700 mg; more preferably 250 mg to 650 mg; and most preferably 300 mg to 600 mg of caproic acid residues.

Typically, the product comprises the oil composition in an amount such that the product comprises from 100 mg to 1000 mg per serving; preferably from 200 mg to 700 mg per serving; more preferably 250 mg to 650 mg per serving; and most preferably 300 mg to 600 mg per serving of caproic acid residues.

It is highly preferred that the nutraceutical product comprises the oil composition in an amount such that the product comprises from 1200 mg to 1600 mg, such as 1200 mg to 1600 mg per serving of a total of butyric acid and caproic acid residues. It is also highly preferred that the nutraceutical product comprises the oil composition in an amount such that the product comprises from 250 mg to 650 mg, such as 250 mg to 600 mg per serving of caproic acid residues.

According to a third aspect of the invention, there is provided a non-therapeutic method of supplementing the diet of an individual, wherein the method comprises administering to the individual an oil composition according to the first aspect of the invention or a nutraceutical product according to the second aspect of the invention.

As discussed above, providing short chain fatty acids to the systemic circulation is associated with various health and wellbeing effects for the individual.

In some instances, the method can comprise maintaining or improving the gastrointestinal function of the individual; maintaining or improving immune system function of the individual; maintaining or improving small intestinal absorption such as postprandial small intestinal absorption in the individual; maintaining or improving blood absorption such as postprandial blood absorption in the individual; regulating the blood glucose level of the individual such as maintaining the blood glucose level of the individual below 7.1 mmol·L⁻¹; preventing or ameliorating diet induced weight gain of the individual; preventing or ameliorating chronic inflammation in the individual; preventing or ameliorating insulin resistance in the individual; and/or maintaining or improving postprandial substrate and energy metabolism in the individual.

Typically, the method comprises administering the oil composition to the individual in an amount such that a daily amount of from 100 mg to 5000 mg; preferably from 500 mg to 2500 mg; more preferably from 750 mg to 1750 mg; and most preferably from 1000 mg to 1750 mg of a total of butyric acid and caproic acid residues is provided by the oil composition to the individual; preferably wherein the daily amount is provided in a single serving.

Most preferably, the method comprises administering the oil composition to the individual in an amount such that a daily amount of from 1200 mg to 1600 mg of a total of butyric acid and caproic acid residues is provided to the individual by the oil composition; preferably wherein the daily amount is provided in a single serving.

Preferably, the method comprises administering the oil composition to the individual in an amount such that a daily amount of from 100 mg to 1000 mg; preferably from 200 mg to 700 mg; more preferably from 250 mg to 650 mg; and most preferably from 300 mg to 600 mg of caproic acid residues is provided to the individual by the oil composition; preferably wherein the daily amount is provided in a single serving.

According to a fourth aspect of the invention, there is provided the use of a triglyceride comprising at least one fatty acid residue comprising from 16 to 18 carbon atoms and at least one caproic acid residue to increase delivery of caproic acid to the systemic circulation from the gastrointestinal tract.

As discussed above, it has been surprisingly found that when caproic acid is included in a triglyceride molecule along with a fatty acid residue comprising from 16 to 18 carbon atoms, the fatty acid having 16 to 18 carbon atoms inhibits the action of gastric lipases present in the stomach which cleave the caproic acid to glycerol bond. This results in the triglyceride proceeding through the gastrointestinal tract where it is then broken down by intestinal lipases. The caproic acid residues are cleaved from the triglyceride in the small intestine where they can then be absorbed by the small intestine and enter the systemic circulation. This does not occur (or at least occurs to a lesser extent) where caproic acid is cleaved from triglycerides in the stomach by gastric lipases since these caproic acid residues are absorbed by stomach cells and used for energy without making it into the systemic circulation. Where triglycerides comprise only caproic acid and other short chain fatty acids, the gastric lipase action is not inhibited and the caproic acid residues are cleaved from the triglycerides in the stomach, thus not finding their way into the systemic circulation which is believed to be necessary for them to provide their health promoting effects. The oil compositions of the invention thus provide improved delivery of caproic acid to the systemic circulation than compositions containing only short chain triglycerides.

The use thus comprises using the triglyceride to increase absorption of caproic acid by the small intestine. The use comprises using the fatty acid residue comprising 16 to 18 carbon atoms to inhibit cleavage of the caproic acid residue from the triglyceride by gastric lipases.

Preferably, the triglyceride comprises at least one fatty acid residue having 18 carbon atoms; and more preferably the triglyceride comprises at least one oleic acid or linoleic acid residue.

Preferably, the triglyceride comprises fatty acid residues having 16 to 18 carbon atoms.

Preferably, the triglyceride is a component of a fat composition according to the first aspect of the invention.

According to a fifth aspect of the invention, there is provided a process for deodorizing an oil composition according to the first aspect of the invention, wherein the process comprises:
(a) providing an oil composition according to the first aspect of the invention; and
(b) deodorizing the oil composition so as to provide a deodorized oil composition.

Preferably, the oil composition in (a) has not previously been deodorized or comprises at least one component that has not previously been deodorized.

Any suitable deodorization conditions known in the art can be used.

Preferably, step (b) comprises carrying out the deodorization step at a temperature of from 100° C. to 200° C.; more preferably from 130° C. to 170° C.; and most preferably from 140° C. to 160° C.

Preferably, the deodorization step is carried out at a pressure of from 0.5 mbar to 8 mbar; more preferably 1 mbar to 6 mbar; and most preferably 1.5 mbar to 3 mbar.

### DESCRIPTION OF THE DRAWINGS

FIG. 1 illustrates plasma concentration of butyric acid residues (C4:0) after consuming different oil compositions of the invention and a control composition.

FIG. 2 illustrates plasma concentration of caproic acid residues (C6:0) after consuming different oil compositions of the invention and a control composition.

FIG. 3 illustrates plasma concentration of GLP-1 after consuming different oil compositions of the invention and a control composition.

FIG. 4 illustrates plasma concentration of glucose after consuming different oil compositions of the invention and a control composition.

FIG. 5 illustrates plasma concentration of insulin after consuming different oil compositions of the invention and a control composition.

FIG. 6 illustrates plasma concentration of TNF-α after consuming different oil compositions of the invention and a control composition.

FIG. 7 illustrates plasma concentration of IFN-γ after consuming different oil compositions of the invention and a control composition.

FIG. 8 illustrates the effect of different concentrations of butyric acid on IL-6 production by stem cell derived adipocytes treated with lipopolysaccharide (LPS). A control conditions without LPS stimulation is included.

FIG. 9 illustrates the effect of different concentrations of hexanoic acid on IL-6 production by stem cell derived adipocytes treated with lipopolysaccharide (LPS). A control condition without LPS stimulation is included.

FIG. 10 illustrates the effect of hexanoic acid, butyric acid, and a combination of butyric acid and hexanoic acid on expression of the cytokine IL-10 in stem cell derived adipocytes treated with lipopolysaccharide (LPS). A condition showing the IL-10 expression without LPS stimulation is included.

### DETAILED DESCRIPTION OF THE INVENTION

The following examples are for illustrative purposes only and are not intended to limit the scope of the invention in any way.

### Example 1

The bioaccessibility and the absorption of three oil compositions A, B and C have been compared using an in vitro TIM-1 model, a computer-controlled dynamic model mimicking the human stomach, small intestine and colon.

Oil composition A is a comparative composition not according to the invention and comprises triglycerides including 71 wt. % by weight of butyric acid residues (C4:0) and from 29 wt. % by weight of caproic acid residues (C6:0).

Oil composition B is according to the invention and is an interesterified oil blend of 8.5 wt. % of oil composition A; 49.0 wt. % of high oleic sunflower oil and 42.5 wt. % of rapeseed oil. Oil composition B comprises 5.6 wt. % of butyric acid residues (C4:0), 2.3 wt. % of caproic acid residues (C6:0) and 68 wt. % of oleic acid residues (C18:1) (as well as other fatty acids).

Oil composition C is a comparative oil composition and comprises medium chain triglycerides (MCT) comprising 59 wt. % octanoic acid residues (C8:0) and 41 wt. % decanoic acid (C10:0) residues. Oil composition C is included as a reference composition for human absorption.

Table 1 shows the results of bioaccessibility and the absorption in the human stomach, small intestine and colon.

TABLE 1

| | | | Oil composition A | | | | | Oil composition B | | | | | Oil composition C | | | | | |
|---|---|---|---|---|---|---|---|---|---|---|---|---|---|---|---|---|---|---|
| | Time (hour) | | 1 | 2 | 3 | 4 | 5 | 1 | 2 | 3 | 4 | 5 | Time (hour) | 1 | 2 | 3 | 4 | 5 |
| Stomach | Cumulative | C4:0 | 52 | 75 | 80 | — | — | 5 | 7 | 7 | — | — | C8:0 | 20 | 35 | 40 | — | — |
| | bioaccessibility | C6:0 | 60 | 90 | 95 | | | 3 | 7 | 7 | — | — | C10:0 | 10 | 15 | 20 | — | — |
| | (wt. %)¹ | | | | | | | | | | | | | | | | | |
| Small | Cumulative | C4:0 | 20 | 56 | 78 | 83 | 85 | 10 | 30 | 40 | 50 | 52 | C8:0 | 10 | 38 | 52 | 58 | 60 |
| | bioaccessibility | C6:0 | 20 | 55 | 75 | 82 | 82 | 10 | 30 | 40 | 50 | 50 | C10:0 | 5 | 38 | 58 | 58 | 58 |
| intestine | (wt. %)² | | | | | | | | | | | | | | | | | |
| Colon | Delivered to | C4:0 | 2 | 9 | 10 | 11 | 11 | 7 | 18 | 18 | 18 | 18 | C8:0 | 2 | 4 | 5 | 6 | 6 |
| | the colon | C6:0 | 3 | 10 | 12 | 12 | 12 | 10 | 20 | 20 | 20 | 20 | C10:0 | 2 | 18 | 26 | 30 | 32 |
| | (wt. %)³ | | | | | | | | | | | | | | | | | |

1. Measured as proportion, out of totally added, of respective fatty acid being in the free form in what leaves the gastric compartment (no absorption/metabolism being simulated in the gastric compartment)
2. Measured as proportion, out of totally added, of respective fatty acid being in the free form in the filtrate over semi-permeable membranes connected to the jejunal and ileal compartments (simulating absorption in the small intestine)
3. Measured as proportion, out of totally added, of respective fatty acid being in the free or bound form in the efflux from the ileum.
The residue residing in the system amounts to the balance to 100% from the sum of what is absorbed in the small intestine and what enters the colon.

### Stomach

The results show that with Oil composition A, about 80-90 wt. % of butyric acid residues (C4:0) and caproic acid residues (C6:0) are released in the stomach. In the human body these free fatty acids will be used as energy in the stomach cells and won't reach the small intestine and the circulation.

The results show that with Oil composition B, 7% of the butyric acid residues (C4:0) and 7% of the caproic acid residues (C6:0) were released from the glycerol backbone in the stomach. The remaining butyric acid and caproic acid residues remain bound in acyl glycerides and are delivered to the small intestine as such.

The results show that with Oil composition C, 40% of the medium chain fatty acid caprylic acid (C8:0) and 20% of capric acid (C10:0) were hydrolysed in the stomach.

The activity of gastric lipase is higher on Oil composition A and Oil composition C than Oil composition B. Oil composition C is a reference oil showing the bioaccessibility and the absorption of fatty acids in the human body. It is believed that the presence of LCFA, i.e. oleic acid residues (C18:1) in the Oil composition B leads to a lower gastric lipase activity on the oil composition. Thus, the majority of SCFA remain on the glycerol backbone to be hydrolysed and absorbed by the small intestine later on.

### Small Intestine and Colon

The results show that lipases in the small intestine are able to cleave caproic acid and butyric acid from the glycerol backbone in the small intestine for all of oil compositions A, B and C.

Whilst it is indicated that after 5 hours, oil composition A has a higher weight percentage of absorbed caproic acid and butyric acid residues than oil composition B, this does not mean that oil composition A provides more caproic acid and butyric acid to the small intestine in a human. For oil composition A, the caproic acid and butyric acid residues are hydrolysed in the stomach prior to reaching the small intestine. The model does not account for the fact that these free caproic acid and butyric acid residues will be used by stomach cells and thus only the hydrolysis and not the metabolism in the stomach is modelled.

For oil composition B, the caproic acids and butyric acids can be cleaved from the glycerol backbones in the small intestine so that they can then be absorbed by the small intestine.

The bioavailability of oil composition B is similar to oil composition C. This means that most of the fatty acids in these compositions are released in the small intestine and can be absorbed into the circulation.

For all the oil compositions, a small portion of the fatty acids reach the colon without uptake. These fatty acids can be used by the microbiota or as an energy source for the colonocytes.

The In vitro TIM-1 model shows that the SCFA in oil composition A are mainly hydrolyzed in the stomach, and not in the small intestine resulting in little SCFA circulating in the human body, meaning that the SCFA are not able to have an effect on metabolic health.

It is believed that the SCFA will be used as an energy source by the stomach cells.

The data also shows that for oil composition B, a significant amount of caproic acid and butyric acid residues are cleaved from triglycerides in the small intestine leading to an increase in blood concentrations of these fatty acids and thus positive systemic effects on metabolic health without the need of colon bacteria.

Furthermore, a greater amount of oil composition B than oil composition A reached the colon which provides positive effects on the microbiota, gastrointestinal health and inflammation.

Additionally, the bioavailability (i.e. intestinal absorption and hence ability to enter the systemic circulation) of butyric acid residues (C4:0) and caproic acid residues (C6:0) in the oil composition B is higher compared to the oil composition A.

### Example 2

Two oil compositions of the invention were produced. These were oil compositions E and F.

The process for manufacturing the oil compositions E and F comprised the steps of:
A). Providing a first starting oil composition comprising 71% by weight of butyric acid residues (C4:0) and 29% by weight of caproic acid residues (C6:0); and a second starting vegetable oil being high oleic sunflower oil, said second starting vegetable oil comprising around 90% by weight unsaturated C16 to C18 fatty acids,
B). Mixing:
   a) either 8.5% by weight of the first starting oil composition and 91.5% by weight of the second starting oil composition to form a mixture, or,
   b) 30% by weight of the first starting oil composition and 70% by weight of the second starting oil composition so as to form a mixture,
C). Performing a chemical interesterification using sodium methoxide,
D). Performing a bleaching using bleaching earth and citric acid,
E). Performing a deodorization at 150° C. so as to obtain a first oil composition 1 (from the mixture obtained in step B) a)) and a second oil composition 2 (from the mixture obtained in step B) b)) and
F). Mixing:
   a) 59.5 wt. % of the first oil composition 1, 31.5 wt. % of the second oil composition 2 and 9 wt. % of a non-interesterified oil being high oleic sunflower oil so as to form an oil composition E, or
   b) 72 wt. % of the second oil composition 2 and 28 wt. % of a non-interesterified oil being high oleic sunflower oil so as to form a oil composition F.

In the first starting oil composition, the butyric acid residues are derived from microbial sources and the caproic acid residues are derived from palm kernel oil.

The different oil compositions produced are summarized below. The fatty acid profile (i.e. composition) was determined by fatty acid methyl ester analysis (FAME) using gas chromatography according to ISO 16958.

Table 2 also includes the fatty acid profile of an oil composition D, which is a control oil composition not according to the invention that is used in the experiments discussed in Example 3 below. Oil composition D was high oleic sunflower oil.

TABLE 2

Vegetable oil compositions (wt. %)

| | Oil | Oil | Oil |
|---|---|---|---|
| | composition D | composition E | composition F |
| C4:0 | <0.1 | 10.0 | 13.2 |
| C6:0 | <0.1 | 5.2 | 7.2 |
| C7:0 | <0.1 | <0.1 | <0.1 |
| C8:0 | <0.1 | <0.1 | <0.1 |
| C10:0 | <0.1 | <0.1 | <0.1 |
| C12:0 | <0.1 | <0.1 | <0.1 |
| C14:0 | <0.1 | <0.1 | <0.1 |
| C14:1 | <0.1 | <0.1 | <0.1 |
| C15:0 | <0.1 | <0.1 | <0.1 |
| C15:1 | <0.1 | <0.1 | <0.1 |
| C16:0 | 4.6 | 3.5 | 3.2 |
| C16:1 | 0.1 | 0.1 | 0.1 |
| C16:2 | <0.1 | <0.1 | <0.1 |
| C17:0 | <0.1 | <0.1 | <0.1 |
| C17:1 | <0.1 | <0.1 | <0.1 |
| C18:0 | 3.0 | 2.4 | 2.3 |
| C18:1 | 80.6 | 69.5 | 66.2 |
| C18:2 | 9.2 | 7.2 | 5.9 |
| C18:3 | 0.3 | 0.2 | 0.1 |
| C18:4 | <0.1 | <0.1 | <0.1 |
| C20:0 | 0.3 | 0.2 | 0.2 |
| C20:1 | 0.2 | 0.3 | 0.2 |
| C20:2 | <0.1 | <0.1 | <0.1 |
| C22:0 | 0.9 | 0.8 | 0.8 |
| C22:1 | 0.2 | 0.2 | 0.2 |
| C24:0 | 0.3 | 0.3 | 0.3 |

In composition E, 44.9% of the caproic acid and butyric acid residues present were present in triglycerides comprising one C16 to C18 fatty acid. 35.9% of the caproic acid and butyric acid residues present were present in triglycerides comprising two C16 to C18 fatty acids. 19.2% of the caproic acid and butyric acid residues present were present in triglycerides comprising three fatty acids, each selected from butyric acid or caproic acid.

In composition F, 53.4% of the caproic acid and butyric acid residues present were present in triglycerides comprising one C16 to C18 fatty acid. 21.0% of the caproic acid and butyric acid residues present were present in triglycerides comprising two C16 to C18 fatty acids. 25.6% of the caproic acid and butyric acid residues present were present in triglycerides comprising three fatty acids, each selected from butyric acid or caproic acid.

### Example 3

A clinical investigation was carried out which included orally administering the oil compositions D, E and F discussed in Example 2 to subjects.

The impact of the orally administrated different oil compositions, especially the bioaccessibility, the absorption and the gastrointestinal health postprandial, was studied on twelve male participants. The participants were either overweight or obese, were aged between 40 and 70 years with a normal fasting glucose (<7.1 mmol/L) and a body mass index (BMI) between 25-35 kg/m². Their blood pressure was within normal range and their weight was stable for at least the last 3 months before the study. The participants did not have any of the following conditions: fasting plasma glucose >7.1 mmol/L, gastroenterological diseases, diabetes mellitus, a history of diabetes mellitus, a history of major abdominal surgery, cardiovascular diseases, cancer, liver or kidney malfunction, disease with a life expectancy of <5 y, alcohol or drug abuse, excessive smoking (>20 cigarettes per day), and exercise training (>3 hours/week). Furthermore, the participants did not follow a hypocaloric, vegan or vegetarian diet, and did not use laxatives, prebiotics, probiotics, or antibiotics for 3 months prior to the study or during the study. The participants also were not using any medication that affects glucose, fat metabolism, and inflammation (including, among others, β-blockers, corticosteroids, and cholesterol-lowering medication). The participants had normoglycemia, and proper kidney and liver functioning.

The participants were investigated on several clinical investigation days (CIDs). On each clinical investigation day, the participants were each administered a meal (a milk shake). The meal was selected so as to provide sufficient energy intake for the 6 h study.

On one of the clinical investigation days, each participant was administered 10 g of the control dosage (composition D) in addition to the meal. On another of the clinical investigation days, each participant was administered 10 g of composition E (i.e. dosage 1) in addition to the milkshake. On another of the clinical investigation days, each participant was administered 10 g of composition F (i.e. dosage 2) in addition to the milkshake.

Composition E (dosage 1) was selected so as to provide a first dose of caproic acid and butyric acid; and composition F (dosage 2) was selected so as to provide a higher dose of caproic acid and butyric acid.

Table 3 shows the amount of butyric acid residues (C4:0) and caproic acid residues (C6:0) in the liquid oil compositions D, E and F.

TABLE 3

| | Dosage | | |
|---|---|---|---|
| | Control dosage | Dosage 1 | Dosage 2 |
| | (composition | (composition | (composition |
| | D) | E) | F) |
| Total SCFA (g) | 2.1 | 3.5 | 4.0 |
| C4:0 (g) | 1.3 | 2.2 | 2.5 |
| C6:0 (g) | 0.8 | 1.3 | 1.5 |
| Added total SCFA (mg) | 0 | 1400 | 1900 |
| Added C4:0 (mg) | 0 | 900 | 1200 |
| Added C6:0 (mg) | 0 | 500 | 700 |

To prevent any potential carry-over effects from a prior clinical investigation day (CID), a wash-out period of at least 1 week (with a maximum of 4 weeks) between CIDs was maintained. The evening before each CID, participants consumed a low fibre standardized meal called “Aviko maaltijdpannetje malse kipfilet” (2.5 g fat, 11.5 g carbohydrates, 4.5 g protein per 100 g of the product). On the CID itself, the participants came in after an overnight fast (>10 hours) and a canula was inserted in an antecubital vein in the elbow to make continuous venous blood sampling possible.

Participants filled in a gastrointestinal Symptom Rating Scale (GSRS) questionnaire to assess gastrointestinal complaints. In the GSRS, participants were asked to rate the presence of any nausea, abdominal pain, abdominal discomfort, bloating, burping/regurgitation, and flatulence on a scale of 0 (not at all) to 5 (a lot). Participants were also able to elaborate on any additional comments concerning gastrointestinal discomfort such as diarrhoea or constipation by the means of an open question. The participants did not suffer any discomfort after consumption of the different oil compositions since the average score for the gastrointestinal complaints was 1.

To determine the effect of butyric acid/caproic acid-enriched triglycerides on substrate metabolism during postprandial conditions and especially the plasma SCFA concentrations, venous blood sampling was repeated at 30, 60, 120, 180, 240, 300 and 360 minutes after consumption of the vegetable oil compositions D, E and F. For SCFA analysis, blood was sampled in a pre-chilled 5 mL heparin tube (Becton Dickinson, NL) since EDTA would interfere with the chromatography results. After collection, all tubes were centrifuged at 3000 rpm, 4° C. for 15 minutes, whereafter plasma was immediately aliquoted, snap-frozen in liquid nitrogen and stored in a −80° C. freezer until analysis.

For SCFA analysis, a portion of 150 μl plasma was used for SCFA determination. An internal standard (IS), 2-Ethylbutyric acid, at a concentration of 36 μM was added. Derivatization was performed by adding 25 μl of a 160 mM 3-Nitrophenylhydrazine (NPH) solution and 25 μl of a 200 mM 1-Ethyl-3-(3-dimethylamino-propyl) carbodiimide hydrochloride solution. This mixture was incubated at 50° C. for 30 minutes. Preparation of the standards curves was done in the same matter as the plasma samples. Concentration range of the standards was 5-400 μM for acetic acid and 0.1-20 μM for the other SCFA. The SCFA-NPH derivatives were extracted by adding diethylether, mixed for 15 minutes and centrifuged at 6000 g for 10 minutes. The upper diethylether layer was collected and dried under a flow of nitrogen gas. Finally, the residue which contains the SCFA-NPH derivatives was dissolved in 60 μl MeOH:water 25:75 (v/v) and directly measured on the liquid chromatography-mass spectrometry (LC-MS). This liquid chromatography was performed using a micro flow high-performance liquid chromatography (HPLC) instrument (Dionex Ultimate 3000) at a flow rate of 200 μl/min. 20 μl of the SCFA extract was loaded onto a Hypersil gold C18 UHPLC column, 10 cm×2.1 mm inner diameter, 1.9 μm particle size (Thermo Fisher Scientific) at 35° C. The mobile phases consisted of solvent A (MeOH:water:FA:AF 10:90:0.1:0.05 v/v) and solvent B (MeOH:water:FA:AF 99:1:0.1:0.05 v/v). The following gradient conditions were applied: 0-2 min isocratic at 25% solvent B. From 2-11 min a linear gradient from 25% to 98% solvent B was applied, from 11-12.5 min isocratic at 98% solvent B to wash away any residual peaks eluting after last peak of interest and from 12.5-16 min isocratic at 25% solvent B to the initial gradient for re-equilibration of the column. The HPLC was coupled on-line to a Q Exactive (Thermo Fisher Scientific) with heated electrospray ionization (HESI) probe (Thermo Fisher Scientific). The mass spectrometer was operated in negative ionization mode. Measurements were done in a targeted selected ion monitoring (tSIM) mode. Resolution was set at 70.000 with a maximum injection time of 200 ms and an isolation window of 4 m/z. The inclusion list was 194.06 m/z for Acetic acid-NPH (C2), 208.07 m/z for Propionic acid-NPH (C3), 222.09 m/z for Butyric acid-NPH (C4), 236.10 m/z for Valeric Acid-NPH (C5) and 250.12 m/z for Caproic acid-NPH (C6). Quantification by integration of the peak areas and plotting each calibration curve was conducted by the workstation Thermo Scientific Xcalibur 4.4 software (Thermo Fisher Scientific).

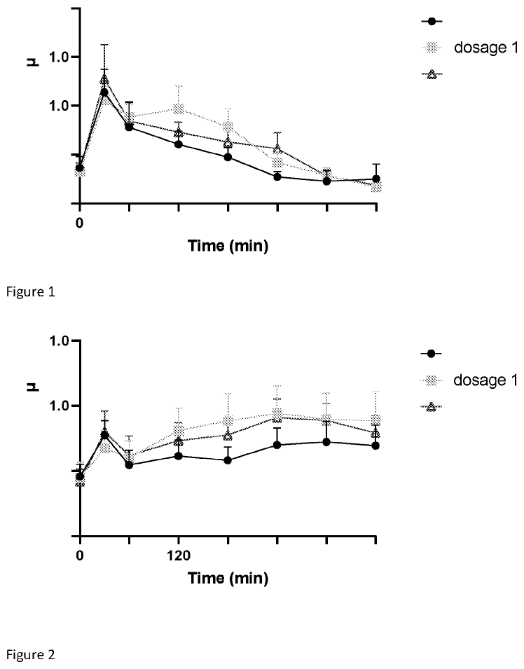

FIG. 1 shows that the blood absorption of butyric acid residues (C4:0) after consuming the oil compositions E and F according to the present invention is higher than the blood absorption of butyric acid residues (C4:0) after consuming the oil composition D. FIG. 2 shows that compositions E and F cause much higher blood absorption of caproic acid (C6:0) than the control composition D.

It can be concluded from the data that intestinal absorption (and thus post prandial plasma concentration) of the SCFA in the small intestine is higher for the compositions of the invention than for control composition D.

It is also interesting that oil composition E showed better absorption of the SCFA into the blood than oil composition F, even though it contained a lower amount of SCFA. Without being limited by theory, it is believed that this may be due to a higher percentage of the SCFA in composition E being present in triglycerides comprising two C16 to C18 fatty acids than composition F. It is thus postulated that where triglycerides comprise one caproic acid residue and two C16 to C18 fatty acid residues, gastric lipases are inhibited from cleaving caproic acid from its glycerol backbone in the stomach to a greater extent than where the triglyceride only comprises one C16 to C18 fatty acid. This finding is unexpected.

Study outcomes included circulating metabolites (glucose, triglycerides, and free fatty acids (FFA)), hormones (insulin, active glucagon-like peptide-1 (GLP-1), and total GLP-1, inflammatory markers (tumor necrosis factor alpha (TNF-α) and interferon gamma (IFN-γ)) were also performed.

To evaluate plasma FFA, triglycerides and glucose in the plasma samples, an automated spectrophotometer (ABX Pentra 400 autoanalyzer, Horiba ABX, Montpellier, France) was used and measured on an automated Cobas Fara spectrophotometric auto-analyzer (Roche Diagnostics, Basel, Switzerland). Circulating plasma insulin was assessed by commercially available radioimmunoassay (RIA) kits (Human Insulin specific RIA, Millipore Corporation, Billerica, MA, USA) and the concentration of inflammatory markers was evaluated by a multiplex enzyme-linked immuno-sorbent assay (Human ProInflammatory II 4-Plex Ultra-Sensitive Kit, Meso Scale Diagnositics, Rockville, MD, USA). Total GLP-1 immunoreactivity was evaluated as described previously using antiserum, which reacts equally with the intact GLP-1 molecule and the primary (N-terminally truncated) metabolite of GLP-1.

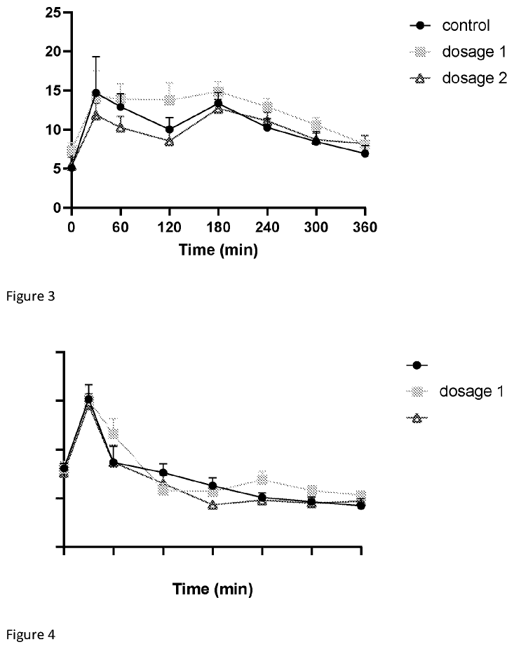

FIG. 3 illustrates that the postprandial response of active glucagon-like peptide-1 (GLP-1), a hormone that may influence appetite and weight regulation is similar after consuming the oil compositions D, E and F.

As illustrated in FIG. 4, the postprandial response of glucose is similar after consuming the oil compositions D, E and F.

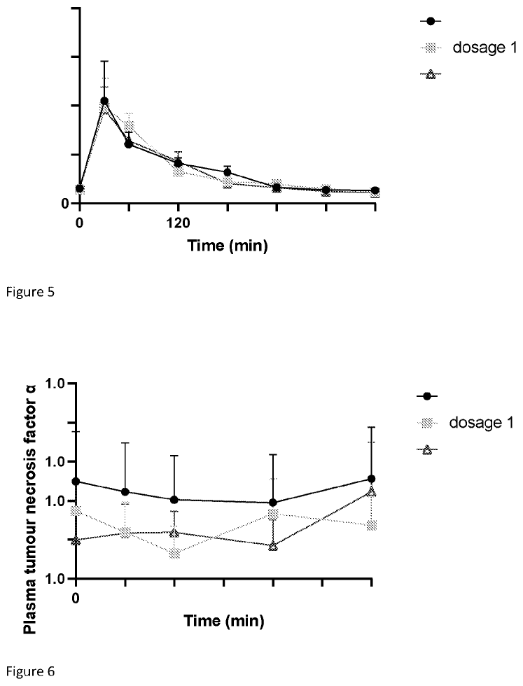

As illustrated in FIG. 5, the postprandial response of insulin is similar after consuming the oil compositions D, E and F.

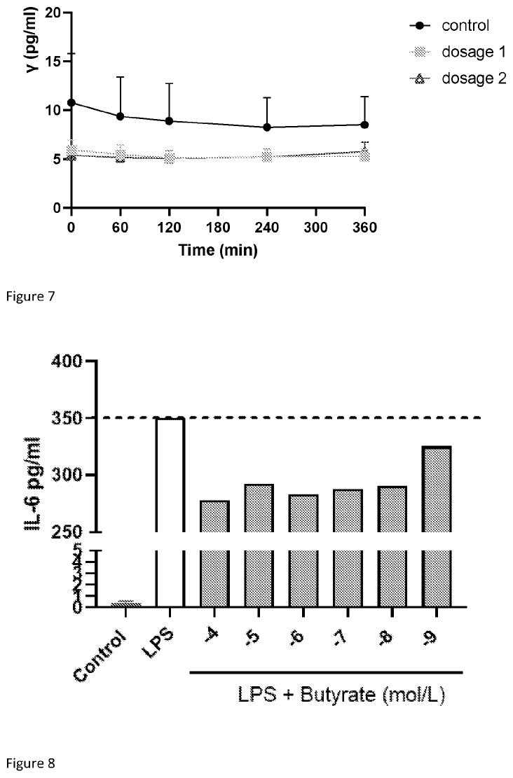

FIGS. 6 and 7 respectively show that the postprandial response of inflammatory markers such as TNF-α and IFN-γ is lower after consuming the oil compositions E and F than when after consuming vegetable oil composition D. This is interesting as it indicates that oil compositions of the invention lower the levels of biomarkers of inflammation circulating in the plasma.

The oil compositions of the invention thus provide an increase in the absorption of SCFA, especially butyric acid residues (C4:0) and caproic acid residues (C6:0), in the small intestine and in the blood of the participants when compared to the control oil composition D. Furthermore, the oil compositions of the invention were well tolerated since their gastrointestinal health was not disturbed. The postprandial response of inflammatory markers was also lowered by the compositions of the invention relative to the control composition. Moreover, the level of glucose in blood was maintained to a healthy level below 7.1 mmol·L-1.

### Example 4

Experiments were carried out to investigate the potential for caproic acid and butyric acid to alleviate (low-grade) inflammation in adipose tissue.

Low-grade inflammation in adipose tissue is known to be mechanistically linked to metabolic disease and organ tissue complications which are considered to be causative of obesity and weight gain in organisms. Low-grade inflammation in adipose tissue can be characterized by increased production of proinflammatory cytokines such as IL-1β, IL-6 and TNFα, and a switch in the phenotype from an anti-inflammatory M2 state to a proinflammatory M1 state.

An experiment was carried out to ascertain the extent to which butyric acid and caproic acid effect the production of the proinflammatory cytokine IL-6 in an inflammatory condition. In the experiment stem cell derived adipocytes were treated with butyric acid and caproic acid after which lipopolysaccharide (LPS) was added to induce inflammation which increased the production of the proinflammatory biomarker IL-6.

### Materials and Method

Pooled human multipotent adipose tissue-derived stem cells (hMADS), were differentiated into the adipogenic lineage, creating a human white adipocyte model. hMADS cells were derived from male human donors with a large range in BMI (20-40 kg/m2) and glucometabolic status, and age between 35 and 70 years of age. Cells were seeded at a density of 2,000 cells/cm2 and kept in a proliferation medium containing Dulbecco's modified Eagle's medium (DMEM), Ham's F-12 Nutrient Mixture (Gibco, Bleiswijk, Netherlands), 10% fetal bovine serum (Bodinco BV, Alkmaar, NL, Netherlands), 50 U/ml penicillin (Gibco), and 50 μg/ml of streptomycin (Gibco)].

At 70-80% confluence, 250 μmol/L IBMX (Sigma, St. Louis, MI, USA) and 5 μmol/L rosiglitazone (Enzo Life Sciences, Raamsdonksveer, Netherlands) were added to induce adipogenic differentiation. Experiments were carried out between days 12 and 14 of the differentiation.

To study the effects of different concentrations of butyric acid and hexanoic acid (10-4 to 10-9 mol) on IL-6 release, hMADS adipocytes were incubated with butyric acid or hexanoic acid at different concentrations. After 24 hours, 5 ug/mL LPS was added for another 24 hours. The supernatants of the cells were collected for IL-6 analysis.

IL-6 cytokine levels in the culture supernatant were evaluated using a electrochemiluminescence (ECL) immunoassay (MSD; Rockville, Maryland) according to the manufacturers protocol.

Three independent identical experiments were performed and the average of these experiments presented.

### Results

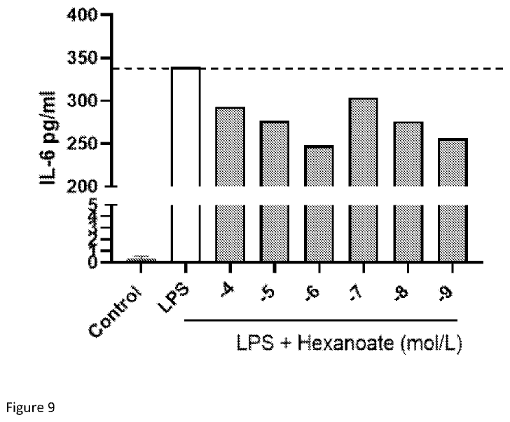

The results are shown in FIG. 8 (butyric acid) and FIG. 9 (hexanoic acid). It can be seen that for both hexanoic acid and butyric acid, at all tested concentrations, the concentration of IL-6 is lower than for the cells treated with LPS but not subsequently treated with these fatty acids. The results thus show that both butyric acid and hexanoic acid reduce concentrations of the proinflammatory cytokine IL-6 in adipose tissues indicating that both tested fatty acids can alleviate (low-grade) inflammation in adipose cells. This indicates that the fatty acids have activity in alleviating the inflammatory processes contributing to obesity and obesity development.

### Example 5

Another experiment was carried out to ascertain the extent to which butyric acid, caproic acid, and a combination of these fatty acids affect expression of the anti-inflammatory biomarker IL-10 in adipose tissue-derived stem cells.

In the experiment, adipose tissue-derived stem cells were pre-treated with butyric acid, caproic acid, and a combination thereof after which lipopolysaccharide (LPS) was added to induce (low-grade) inflammation. LPS-incubation decreased the expression of the anti-inflammatory biomarker IL-10.

### Materials and Method

Pooled human multipotent adipose tissue-derived stem cells (hMADS), were differentiated into the adipogenic lineage, creating a human white adipocyte model. hMADS cells were derived from male human donors with a large range in BMI (20-40 kg/m2) and glucometabolic status, and age between 35 and 70 years of age. Cells were seeded at a density of 2,000 cells/cm2 and kept in proliferation medium containing Dulbecco's modified Eagle's medium (DMEM), Ham's F-12 Nutrient Mixture (Gibco, Bleiswijk, Netherlands), 10% fetal bovine serum (Bodinco BV, Alkmaar, NL, Netherlands), 50 U/ml penicillin (Gibco), and 50 μg/ml of streptomycin (Gibco)].

At 70-80% confluence, 250 μmol/L IBMX (Sigma, St. Louis, MI, USA) and 5 μmol/L rosiglitazone (Enzo Life Sciences, Raamsdonksveer, Netherlands) were added to induce adipogenic differentiation. Experiments were carried out between days 12 and 14 of the differentiation.

To study the effect of butyric acid (10⁻⁴M) and hexanoic acid (10⁻⁵M) on the expression of the anti-inflammatory cytokine IL-10, hMADS derived adipocytes were incubated with butyric acid, hexanoic acid or the combination thereof. After 24 hours 5 ug/mL LPS was added for another 24 hours. The cells were collected for IL-10 and IL-6 analysis.

To determine the IL-10 mRNA expression, total RNA was extracted from the hMADS derived adipocytes using TRIzol reagent (Invitrogen) and SYBR-Green. Real-time PCRs were performed using an iCylcer (Biolegio, Nijmegen, The Netherlands, primer sequence). Results were normalized for 18S ribosomal RNA (calculating delta-delta Ct values).

Two independent identical experiments were performed, and the average of the experiments is presented.

### Results

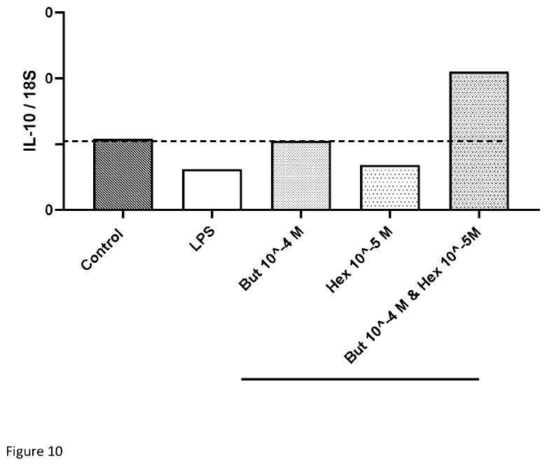

The results of the experiment are shown in FIG. 10. It was found that LPS decreases the expression of the anti-inflammatory cytokine IL-10. The tested butyric acid and hexanoic acid concentrations both showed a small increase in expression of IL-10 relative to the sample treated with LPS but no short chain fatty acids. This indicates that both tested short chain fatty acids increase expression of the anti-inflammatory cytokine IL-10 which indicates both fatty acids are associated with prevention or alleviation of low-grade inflammation of adipose tissue. This indicates that the fatty acids have activity in alleviating or preventing obesity.

Surprisingly, a large and synergistic increase in IL-10 expression was observed in the sample treated with both butyric acid and hexanoic acid. This increase in IL-10 expression is an increase of a factor of two over the expression levels for each fatty acid alone. These results indicate that the combination of butyric acid and caproic acid can be used to provide a desirable and synergistic increase in anti-inflammatory biomarkers indicating that the combination of these fatty acids could have a synergistic effect in preventing or ameliorating the inflammatory effects in obesity.

1. An oil composition comprising from 1% to 20% by weight of caproic acid residues (C6:0); at least 55% by weight of C16 to C18 unsaturated fatty acid residues; and less than 40% by weight of CS to C24 saturated fatty acid residues; wherein said percentages of fatty acid residues refers to fatty acids bound as acyl groups in glycerides in the oil composition and being based on the total weight of saturated and unsaturated C4 to C24 fatty acid residues bound as acyl groups present in the oil composition; and wherein at least 50% of the caproic acid residues present in the oil composition are present in a triglyceride comprising at least one caproic acid residue and at least one fatty acid residue comprising from 16 to 18 carbon atoms.

2. An oil composition according to claim 1, wherein the oil composition comprises from 2% to 30% by weight of butyric acid residues (C4:0); and wherein at least 50% of the butyric acid residues present in the oil composition are present in a triglyceride comprising at least one butyric acid residue and at least one fatty acid residue comprising from 16 to 18 carbon atoms.

3. An oil composition according to claim 1, wherein the oil composition comprises butyric acid (C4:0) in an amount of from 2% to 25% by weight.

4. An oil composition according to claim 1, wherein the oil composition comprises butyric acid (C4:0) in an amount of from 7.5% to 30% by weight.

5. An oil composition according to claim 1, wherein the oil composition comprises caproic acid (C6:0) in an amount of from 1% to 15% by weight.

6. An oil composition according to claim 1, wherein the oil composition comprises caproic acid (C6:0) in an amount of from 2% to 10% by weight.

7. An oil composition according to claim 1, wherein the oil composition comprises caproic acid (C6:0) in an amount of from 5% to 20% by weight.

8. An oil composition according to claim 1, wherein at least 60% of the caproic acid residues present in the oil composition; and at least 60% of butyric acid residues present in the oil composition are present in a triglyceride comprising at least one butyric acid or caproic acid residue and at least one fatty acid residue comprising from 16 to 18 carbon atoms.

9. An oil composition according to claim 1, wherein at least 70% of the caproic acid residues present in the oil composition and at least 70% of the butyric acid residues present in the oil composition are present in a triglyceride comprising at least one butyric acid or caproic acid residue and at least one fatty acid residue comprising from 16 to 18 carbon atoms.

10. An oil composition according to claim 1, wherein at least 80% of the caproic acid residues present in the oil composition; and at least 80% of the butyric acid residues present in the oil composition are present in a triglyceride comprising at least one butyric acid or caproic acid residue and at least one fatty acid residue comprising from 16 to 18 carbon atoms.

11. An oil composition according to claim 1, wherein at least 20% of the caproic acid residues present in the oil composition; and at least 20% of butyric acid residues present in the oil composition are present in a triglyceride comprising a butyric acid or caproic acid residue and two fatty acid residues comprising from 16 to 18 carbon atoms.

12. An oil composition according to claim 1, wherein at least 25% of the caproic acid residues present in the oil composition; and at least 25% of the butyric acid residues present in the oil composition are present in a triglyceride comprising a butyric acid or caproic acid residue and two fatty acid residues comprising from 16 to 18 carbon atoms.

13. An oil composition according to claim 1, wherein at least 50% of the caproic acid residues present in the oil composition; and at least 50% of the butyric acid residues present in the oil composition are present in a triglyceride comprising a butyric acid or caproic acid residue and two fatty acid residues comprising from 16 to 18 carbon atoms.

14. An oil composition according to claim 1, wherein the oil composition has a weight ratio of butyric acid residues (C4:0) to caproic acid residues (C6:0) of from 1:1 to 6:1.

15. An oil composition according to claim 1, wherein the oil composition comprises C16 to C18 unsaturated fatty acid residues in an amount of at least 60% by weight.

16. An oil composition according to claim 1, wherein the oil composition comprises fatty acid residues having from 16 to 18 carbon atoms in an amount of from 60% to 95% by weight.

17. An oil composition according to claim 1, wherein the oil composition has a weight ratio of fatty acid residues having from 16 to 18 carbon atoms to butyric acid residues (C4:0) and caproic acid residues (C6:0) of from 1:1 to 20:1.

18. An oil composition according to claim 1, wherein the oil composition comprises 10% by weight or less of palmitic acid (C16:0).

19. An oil composition according to claim 1, wherein the oil composition comprises 10% by weight or less of a total amount of fatty acid residues having from 16 to 17 carbon atoms.

20. An oil composition according to claim 1, wherein the oil composition comprises from 10% to 40% by weight of total saturated fatty acids.

21. An oil composition according to claim 1, wherein the oil composition comprises 10% by weight or less of stearic acid residues (C18:0).

22. An oil composition according to claim 1, wherein the oil composition comprises from 56% to 85% by weight, of oleic acid residues (C18:1) and/or 1% to 10% by weight of linoleic acid residues (C18:2).

23. An oil composition according to claim 1, wherein the oil composition comprises less than 5% by weight of trans unsaturated fatty acids.

24. An oil composition according to claim 1, wherein the oil composition is a non-hydrogenated oil composition.

25. An oil composition according to claim 1, wherein the oil composition comprises a component produced by interesterification, transesterification or esterification.

26. An oil composition according to claim 1, wherein the oil composition comprises an interesterified blend, wherein the blend comprises (a) a vegetable oil selected from sunflower oil, rapeseed oil, canola oil, safflower oil, soybean oil, algae oil, olive oil, high oleic acid versions thereof, or mixtures thereof, and (b) a triglyceride oil comprising at least 10% by weight of caproic acid residues.

27. An oil composition according to claim 26, wherein the triglyceride oil (b) comprises at least 20% by weight of caproic acid residues and from 60% to 80% by weight of butyric acid residues.

28. An oil composition according to claim 26, wherein the interesterified blend is an interesterified blend of from 60% to 95% by weight of vegetable oil (a) and from 5% to 40% by weight of triglyceride oil (b).

29. An oil composition according to claim 26, wherein the oil composition further comprises a vegetable oil component (c) that has not been produced by interesterification, transesterification or esterification.

30. An oil composition according to claim 29, wherein the vegetable oil composition (c) is present in the oil composition in an amount of from 1% to 35% by weight; and wherein the interesterified blend is present in the oil composition in an amount of from 65% to 99% by weight.

31. An oil composition according to claim 1, wherein at least 50% of the fatty acid residues present in the oil composition are non-animal derived.

32. An oil composition according to claim 1, wherein the oil composition is a deodorized oil composition.

33. A nutraceutical product comprising an oil composition according to claim 1.

34.-39. (canceled)

40. A non-therapeutic method of supplementing the diet of an individual, wherein the method comprises administering to the individual an oil composition according to claim 1.

41.-50. (canceled)

51. A process for deodorizing an oil composition, wherein the process comprises:
(a) providing an oil composition according to claim 1; and
(b) deodorizing the oil composition so as to provide a deodorized oil composition.

52.-53. (canceled)
